(19) United States
(12) Patent Application Publication
(10) Pub. No.: US 2004/0267316 A1
(43) Pub. Date: Dec. 30, 2004

(54) Liquid crystal polymer/polyester composite high strength suture

(75) Inventors: Amber Powell, St. Petersburg, FL (US); Drew Amery, Clearwater, FL (US)

Correspondence Address: H. JAY SPIEGEL, P.O. Box 11, Mount Vernon, VA, 22121, US

(73) Assignee: LINVATEC CORPORATION

(21) Appl. No.: 10/874,302

(22) Filed: Jun. 24, 2004

Related U.S. Application Data
(60) Provisional application No. 60/482,994, filed on Jun. 27, 2003.

(51) Int. Cl. A61B 17/06; A61L 17/00; A61B 17/04

(52) U.S. Cl. 606/231; 606/228; 606/224

(57) ABSTRACT

A high strength suture is made from a LCP/polyester composite of braided filaments having a high coefficient of friction. Knots tied in a suture made from such braided filaments have a tendency to stay in place better than the prior art UHMWPE/polyester composite knots. The inventive suture may include a multifilament cover of fibers of LCP and/or LCP composite fibers. Where a core is employed, the core may be made up of twisted fibers of LCP and liquid crystal composite fibers. If desired, the composite fibers can be a combination of LCP and other polymers such as polyester, nylon, fluoropolymers such as FEP or PTFE or olefins such as polypropylene, polyethylene or UHMWPE. A low friction coating, such as silicone, may be applied over the outside surfaces of the suture.

### BACKGROUND OF THE INVENTION

1. Field of the Invention

This invention relates to medical grade sutures. More particularly, it refers to high strength sutures made from a composite of a liquid crystal polymer/polyester composite of braided filaments.

2. Background of the Prior Art

There is a need for a medical grade, high strength suture for use in orthopedic procedures such as rotator cuff repair, Achilles tendon repair, patellar tendon repair, ACL/PCL reconstructions, hip and shoulder reconstructions, and also to replace the standard polyester suture currently used in suture anchors. The high strength suture also could be attached to a curved needle or soft tissue anchor. Standard nonabsorbable multifilament sutures are typically composed of braided polyester or nylon. These sutures typically have a U.S. Pharmacopia (USP) knot break strength of under 16 pounds. Liquid Crystal Polymers (LCP) are a class of high performance polymers that have excellent dimensional stability, strength, and rigidity, as well as temperature and chemical resistance. Vectran® is a Trademark associated with one of these high strength Liquid Crystal Polymers (LCP) that is melt spun into fibers by Celanese®.

High strength sutures on the market are composed of a composite of ultra high molecular weight polyethylene (UHMWPE) (Dyneema™ or Spectra™) and polyester. While such sutures fulfill the strength requirement, they are lacking in the other properties that need to be considered. Some of these properties include, but are not limited to, knot slidability, elongation, handling ability, knot staying capabilities, ultimate failure of a knotted loop, all in addition to the knot break strength. Current high strength polyethylene/polyester sutures are deficient in some of these areas due to the inherent properties of the ultra high molecular weight polyethylene (UHMWPE). UHMWPE has an extremely low coefficient of friction, comparable to Teflon®. This hinders the ability of the knot to stay in place once tied down. A high strength suture having better handling properties is needed.

### SUMMARY OF THE INVENTION

The present invention relates to the discovery of a high strength suture made from a LCP/polyester composite of braided filaments. The present invention includes the following interrelated objects, aspects and features:

(1) In the preferred embodiment of the present invention, a suture is made of an LCP filament, such as that which is referred to by the Trademark Vectran®, made from a wholly aromatic polyester containing 4-hydroxybenzoic acid units and 2,6-hydroxynaphthoic acid units, and which has a relatively high coefficient of friction as compared to UHMWPE.

(2) Knots tied with a Vectran®/polyester braided filament have a tendency to stay in place better than the prior art UHMWPE/polyester composite knots.

(3) The suture of this invention in the preferred embodiment thereof includes a multifilament cover of fibers of LCP and/or LCP composite fibers.

(4) Where a core is employed, the core may be made up of twisted fibers of LCP and liquid crystal composite fibers. If desired, the composite fibers can be a combination of LCP and other polymers such as polyester, nylon, fluoropolymers such as FEP or PTFE or olefins such as polypropylene, polyethylene or UHMWPE.

(5) In the preferred embodiment, the ratio of LCP filaments to non-LCP filaments is 5% to 95% by weight.

(6) In the present invention, the cover consisting of multiple braided filaments of LCP can vary between 4 and 24 filaments in each braided component. If desired, a silicone coating can be provided over the suture to ease traversing of body soft tissues.

(7) The suture may be attached to a needle or to a soft tissue anchor.

As such, it is a first object of the present invention to provide a liquid crystal polymer/polyester composite high strength suture.

It is a further object of the present invention to provide such a suture which includes a relatively high coefficient of friction so that knots tied using the suture tend to remain tied.

It is a still further object of the present invention to provide such a suture which includes a multifilament cover of fibers of LCP and/or LCP composite fibers.

It is a yet further object of the present invention to provide such a suture with the option of a core made up of twisted fibers of LCP and liquid crystal composite fibers.

It is a still further object of the present invention to provide such a suture, in which the core is made up of twisted fibers of LCP and liquid crystal composite fibers, with composite fibers consisting of a combination of LCP and one or more other polymers chosen from the group consisting of polyester, nylon, fluoropolymers such as FEP or PTFE or olefins such as polypropylene, polyethylene or UHMWPE.

These and other objects, aspects and features of the present invention will be better understood from the following detailed description of the preferred embodiments when read in conjunction with the appended drawing figure.

### BRIEF DESCRIPTION OF THE DRAWINGS

The invention can be best understood by those having ordinary skill in the art by reference to the following detailed description when considered in conjunction with the accompanying drawing, in which:

FIG. 1 is a cross-sectional view of a representative suture consisting of braided filaments around a core, according to this invention.

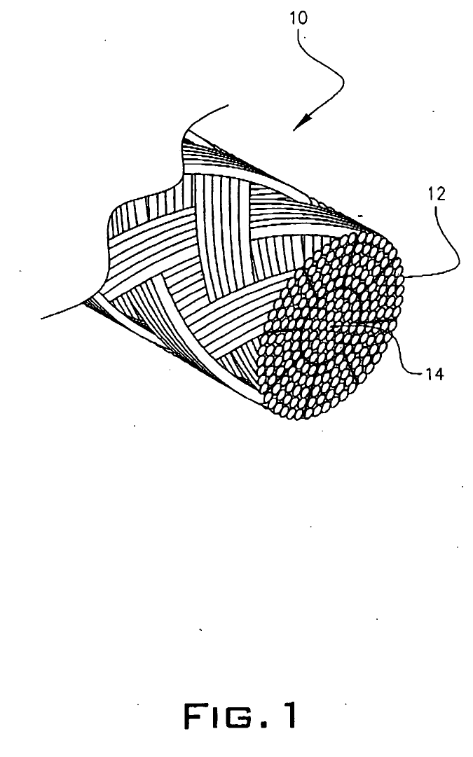

### DETAILED DESCRIPTION OF THE INVENTION

Various embodiments of the present invention will be discussed with reference to the appended drawing. This drawing depicts only an illustrative embodiment of the invention and is not to be considered limiting of the scope of the present invention.

The sutures 10 of this invention contain a core 14 of twisted fibers of LCP and liquid crystal composite fibers. The composite fibers can be a combination of LCP and other polymers such as polyester, nylon, fluoropolymers such as FEP or PTFE or olefins such as polypropylene, polyethylene or UHMWPE. The core provides resistance to flattening, as well as increased strength of the suture. The ratio of LCP filaments to non-LCP filaments is 5 to 95% by weight. The cover 12 of multiple braided filaments of LCP can vary between 4 and 24 filaments in each braided component. In addition, the suture 10 can be coated with silicone or like material used in suture coatings, to better facilitate traversing soft body tissue. However, even with the silicone or like material coating, the coefficient of friction of the suture, in accordance with the teachings of the present invention, is still significantly higher than those of UHMWPE or TEFLON® to ensure that the objects of the present invention are achieved, particularly the object of being able to tie a knot with the inventive suture, whereby the knot is unlikely to loosen. The suture 10 can be made to any USP size including number 2.

The braids of the invention are made using conventional braiding technology and equipment used in the medical industry for preparing multifilament sutures. The method of formation of filament from LCP is set forth in U.S. Pat. No. 4,606,875, incorporated herein by reference.

Alternatively, the braids could be woven in a spiral or in lattices as described in U.S. Pat. Nos. 4,959,069 and 5,059,213, the disclosures of which are incorporated herein by reference.

Tests on an initial Vectran®/polyester prototype suture resulted in a knot break strength in excess of 20 pounds. A preferred knot break strength is around 25 pounds. The minimum USP requirement for knot break strength of a number 2 suture as determined by the USP is 7.76 pounds. These tests were done on a MTS using a 220 pound load cell and following the USP Ultimate Knot-Pull Tensile Strength Test.

As such, an invention has been disclosed in terms of preferred embodiments thereof which fulfill each and every one of the objects of the invention as set forth hereinabove, and provide a new and useful liquid crystal polymer/polyester composite high strength suture of great novelty and utility.

Of course, various changes, modifications and alterations in the teachings of the present invention may be contemplated by those skilled in the art without departing from the intended spirit and scope thereof.

As such, it is intended that the present invention only be limited by the terms of the appended claims.

1. A high strength suture for use in orthopedic procedures comprising:
a) a core of twisted fibers of either (1) a Liquid Crystal Polymer (LCP) or (2) a composite of liquid crystal and other polymers selected from the group consisting of polyester, polyamide, fluoropolymer, and olefins having alkyl groups of two to three carbon atoms;
b) a cover around the core of braided multifilaments made of LCP or composite LCP;
c) the suture having a ratio of liquid crystal filaments to non-liquid crystal filaments of about 5 to 95% by weight and a knot break strength in excess of twenty (20) pounds.

2. The suture of claim 1, attached to a needle.

3. The suture of claim 1, attached to a soft tissue anchor.

4. A high strength suture for use in orthopedic procedures comprising:
a) a core of twisted fibers of either (1) a Liquid Crystal Polymer (LCP) or (2) a composite of liquid crystal and other polymers selected from the group consisting of polyester, polyamide, fluoropolymer, and olefins having alkyl groups of two to three carbon atoms;
b) a cover around the core of braided multifilaments made of LCP or composite LCP;
c) the suture having a ratio of liquid crystal filaments to non-liquid crystal filaments of 5 to 95% by weight and a knot break strength in excess of twenty (20) pounds;
d) a low friction coating over said suture to facilitate passage through tissue.

5. The suture of claim 4, attached to a needle.

6. The suture of claim 4, attached to a soft tissue anchor.

7. The suture of claim 4, wherein said low friction coating is made of silicone.

8. The suture of claim 5, wherein said low friction coating is made of silicone.

9. The suture of claim 6, wherein said low friction coating is made of silicone.

10. The suture of claim 1, wherein said LCP comprises a wholly aromatic polyester containing 4-hydroxybenzoic acid units and 2,6-hydroxynaphthoic acid units.

11. The suture of claim 4, wherein said LCP comprises a wholly aromatic polyester containing 4-hydroxybenzoic acid units and 2,6-hydroxynaphthoic acid units.

12. A high strength suture comprising a plurality of twisted fibers at least some of which are made of a liquid crystal polymer (LCP).

13. The suture of claim 12, wherein the LCP comprises a wholly aromatic polyester containing 4-hydroxybenzoic acid units and 2,6-hydroxynaphthoic acid units.

14. The suture of claim 12, including a core of twisted fibers and a cover around the core.

15. The suture of claim 14, wherein said twisted fibers of said core include a composite of LCP and other polymers selected from the group consisting of polyester, polyamide, fluoropolymer, and olefins having alkyl groups of two to three carbon atoms.

16. The suture of claim 15, wherein, with regard to said composite, a ratio of LCP filaments to non-LCP filaments is in the range of about 5% to 95%.

17. The suture of claim 12, attached to a needle.

18. The suture of claim 12, attached to a soft tissue anchor.

19. The suture of claim 17, coated with a low friction coating.

20. The suture of claim 18, coated with a low friction coating.
